(19) United States
(12) Patent Application Publication
(10) Pub. No.: US 2025/0312136 A1
(43) Pub. Date: Oct. 9, 2025

(54) Graft-Integrated Intravascular Catheter Fixation

(71) Applicant: Abiomed, Inc., Danvers, MA (US)

(72) Inventors: Christopher Sheils, Danvers, MA (US); Kevin Ray, Danvers, MA (US)

(73) Assignee: Abiomed, Inc., Danvers, MA (US)

(21) Appl. No.: 19/087,732

(22) Filed: Mar. 24, 2025

Related U.S. Application Data
(60) Provisional application No. 63/575,274, filed on Apr. 5, 2024.

(51) Int. Cl. A61F 2/07

(52) U.S. Cl. CPC A61F 2/07; A61F 2002/075

(57) ABSTRACT

A graft provided with a catheter fixation device for securing a catheter within a cardiovascular system of a patient. The graft having a proximal end, a distal end, and a sidewall defining a conduit having length extending between the proximal end and the distal end. A fixation device integrated into the graft, the fixation device being configured to transition a portion of the conduit from a first diameter, in which a catheter is movable along the length of the conduit, to a second diameter smaller than the first diameter, in which the catheter is fixed within the conduit. A method of delivering and stabilizing a medical device within a cardiovascular system of a patient is also provided.

### CROSS-REFERENCE TO RELATED APPLICATIONS

This application claims the benefit of the filing date of U.S. Provisional Patent Application No. 63/575,274 filed Apr. 5, 2024, the disclosure of which is hereby incorporated herein by reference.

### BACKGROUND OF THE DISCLOSURE

The present disclosure relates to catheter fixation devices, and more particularly, to a fixation device integrated into a graft that allows a physician to stabilize a catheter within the cardiovascular system of a patient.

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the IMPELLA® family of devices designed by Abiomed, Inc. of Danvers, MA. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### BRIEF SUMMARY OF THE DISCLOSURE

In accordance with a first aspect of the present disclosure, a graft integrated with a catheter fixation device is provided. Among other advantages, the graft is designed to be subcutaneously implanted into a patient and utilized to secure a catheter carrying an intracardiac pump. Securing the catheter subcutaneously reduces the distance between the securement device and the target site (e.g., across a native heart valve) compared to traditional securement devices which are externally secured to skin of the patient. As a result, the graft disclosed herein, reduces the length between the catheter fixation device and the distal end of the catheter, which in turn reduces the likelihood of migration of the catheter compared to the traditional catheter fixation devices.

One embodiment of the graft-integrated fixation device may include a graft having a proximal end, a distal end, and a sidewall defining a conduit having length extending between the proximal end and the distal end. The fixation device may be integrated into the graft and configured to transition a portion of the conduit from a first diameter, in which a catheter is movable along the length of the conduit, to a second diameter smaller than the first diameter, in which the catheter is fixed within the conduit.

The fixation device may include a filament, and the filament may be weaved within a portion of the sidewall. In some embodiments, the filament may be arranged as a draw-string configured to cinch the portion of the conduit.

In another embodiment, the fixation device may be a clamp. The clamp may include an inner ring formed of a resilient material and wings formed of a rigid material configured to clamp the inner ring. In some aspects, the wings may be clamped via a securement device such as a staple, sutures, or the like.

In another embodiment, an assembly is provided. The assembly may include a catheter for delivering a medical device into a cardiovascular system of a patient and a graft. The graft may include a proximal end, a distal end, a sidewall defining a conduit having a length extending between the proximal end and the distal end, and a fixation device integrated into the graft. The fixation device may be configured to transition a portion of the conduit from a first diameter, in which the catheter is movable along the length of the conduit, to a second diameter smaller than the first diameter, in which the catheter is fixed within the conduit.

The assembly may include a medical device in the form of an intracardiac pump. In some embodiments, the fixation device may include a filament, for example, a filament that is weaved within a portion of the sidewall of the graft. The filament may be arranged as a draw-string configured to cinch the portion of the conduit.

In another embodiment, the fixation device may include a clamp having an inner ring formed of a resilient material and an outer rigid material arranged to clamp the inner ring. The outer rigid material may include first and second wings and/or define one or more apertures configured to receive a fastening device, for example, a staple or a suture.

A method of stabilizing a medical device within a cardiovascular system of a patient is also provided herein. The method may include the steps of: tracking a catheter, carrying a medical device, through a conduit of a graft integrated with a fixation device and into the cardiovascular system of the patient; positioning the medical device at a target site within the cardiovascular system of the patient; and actuating the fixation device to transition a portion of the conduit from a first diameter, in which the catheter is movable along a length of the conduit, to a second diameter smaller than the first diameter, in which the catheter is fixed within the conduit and relative to the cardiovascular system of the patient.

The medical device may be an intracardiac pump and the target site may be across a native heart valve. The method may further include the step of creating an incision through skin of the patient adjacent a clavicle of the patient.

In some embodiments, the fixation device may include a filament, and the actuating step may include tensioning the filament to cinch the portion of the graft. In other embodiments, the fixation device may include a resilient ring and an outer rigid material, and the actuating step may include applying a clamping force to the outer rigid material. In some aspects, the clamping force may be applied by securing a staple through one or more apertures of the outer rigid material, which compresses the resilient ring about the catheter.

### BRIEF DESCRIPTION OF THE DRAWINGS

Various embodiments of the present disclosure are described herein with reference to the drawings, wherein:

FIG. 1 is a highly schematic cutaway view of the human heart, showing a percutaneous axillary artery puncture approach for delivering an intracardiac heart pump across the aortic valve;

FIG. 2 is perspective view of an intracardiac heart pump and sheath assembly according to an embodiment of the present disclosure;

FIG. 3 is a highly schematic cutaway view of the human showing the intracardiac heart pump of FIG. 2 properly positioned across the native aortic valve;

FIG. 4 is a perspective view of a graft integrated with a fixation device in the form of a filament in accordance with an embodiment of the present disclosure;

FIG. 5 is a perspective view of a graft integrated with a fixation device in the form of a clamp in accordance with another embodiment of the present disclosure; and

FIG. 6 is a cross-section view of the graft of FIG. 5 taken along line A-A.

### DETAILED DESCRIPTION

Intracardiac pump assemblies can be introduced into the heart either surgically or percutaneously and used to pump blood from one location in the heart or circulatory system to another location in the heart or circulatory system. When deployed in the heart, for example, an intracardiac pump can transfer blood from the left ventricle to the aorta, or from the inferior vena cava to the pulmonary artery. Traditionally, intracardiac pumps operate in parallel with the native heart to supplement cardiac output and partially or fully unload components of the heart. For this reason, intracardiac pumps are often utilized in instances of cariogenic shock, during high-risk percutaneous coronary intervention (PCI), right heart failure, congestive heart failure, or severe lung failure, to relieve stress on the heart during recovery or while the patient awaits a heart transplant. Examples of such systems include the IMPELLA® family of devices designed by Abiomed, Inc., Danvers Mass.

Several IMPELLA® devices are of relatively small circumferential size and can be percutaneously delivered into a patient less invasively than intracardiac pumps that are implanted during traditional, full open-chest surgery. During delivery, the catheter is tracked through the vasculature of a patient to advance the intracardiac pump to a target site (e.g., across the aortic valve in the case of “left-side” intracardiac devices, or across the pulmonary valve in the case of “right-side” intracardiac devices). Echocardiography, fluoroscopy, and/or other imaging techniques may be utilized during delivery to properly position the intracardiac pump. Once in position, the intracardiac pump may be turned on to suction blood through an inflow portion (e.g., a blood inflow cage) of the intracardiac device, and to expel the pumped blood from an outflow portion (e.g., a blood outflow cage) of the intracardiac device. In the case of left-side intracardiac pumps, the blood inflow cage of the intracardiac device may be positioned within the left ventricle of the heart, and the blood outflow cage of the intracardiac pump may be positioned within the aorta. On the other hand, in the case of right-side intracardiac pumps, the blood inflow cage of the intracardiac device may be positioned within the inferior vena cava, and the blood outflow cage of the intracardiac device may be positioned within the pulmonary artery.

The clinical success of intracardiac pumps is dependent, in part, on proper positioning of the pump. For example, in the case of left-side intracardiac pumps, if the blood inflow cage is not properly positioned within the left ventricle, and without obstruction from anatomical structures such as the ventricular wall or the mitral valve, the intracardiac pump will inefficiently suction blood from the left ventricle. Additionally, if the blood outflow cage of the intracardiac pump is not sufficiently positioned within the aorta, the pumped blood may return to the left ventricle, causing further stress on the heart. Thus, it is important for intracardiac pumps to be stabilized in the proper position throughout the entire treatment. For this reason, delivery devices used in percutaneous intracardiac pump procedures, often include a butterfly structure that can be secured to the skin of a patient and a securement device, such as a conventional Tuohy-Borst type device, to prevent the catheter from moving after the intracardiac pump has been properly positioned.

Despite the improvements that have been made to intracardiac pumps and associated delivery assemblies, shortcomings remain. For example, the inventors have recognized that even when the assembly is sutured or otherwise secured to the skin of the patient, ambulation or other movement of the patient, can alter the position of the catheter relative to the anatomy of the patient and/or the access site, and cause the intracardiac pump to migrate from the desired position (i.e., become mispositioned). In some instances, such movement may even damage or completely break the sutures, resulting in further movement of the unsecured catheter. Accordingly, the inventors have recognized and appreciated the numerous benefits associated with integrating a fixation device into a graft through which the catheter is delivered to prevent the catheter from migrating relative to cardiovascular system of the patient and/or the access site.

When deployed in the heart, an intracardiac device collects blood from one area of the heart and pumps the blood to another area of the heart, to assist the heart in performing its normal function. As used herein in connection with an intracardiac pump, the term “inflow” refers to the portion of the intracardiac pump through which blood enters the pump, and the term “outflow” refers to the portion of the intracardiac pump through which blood is expelled. When used in connection with devices for delivering the intracardiac pump into a patient, the terms “proximal” and “distal” are to be taken as relative to the user of the delivery devices. For example, “proximal” or “proximal end” is to be understood as relatively close to the operator, and “distal” or “distal end” is to be understood as relatively farther away from the operator. Also as used herein, the terms “substantially,” “generally,” “approximately,” and “about” are intended to mean that slight deviations from absolute are included within the scope of the term so modified.

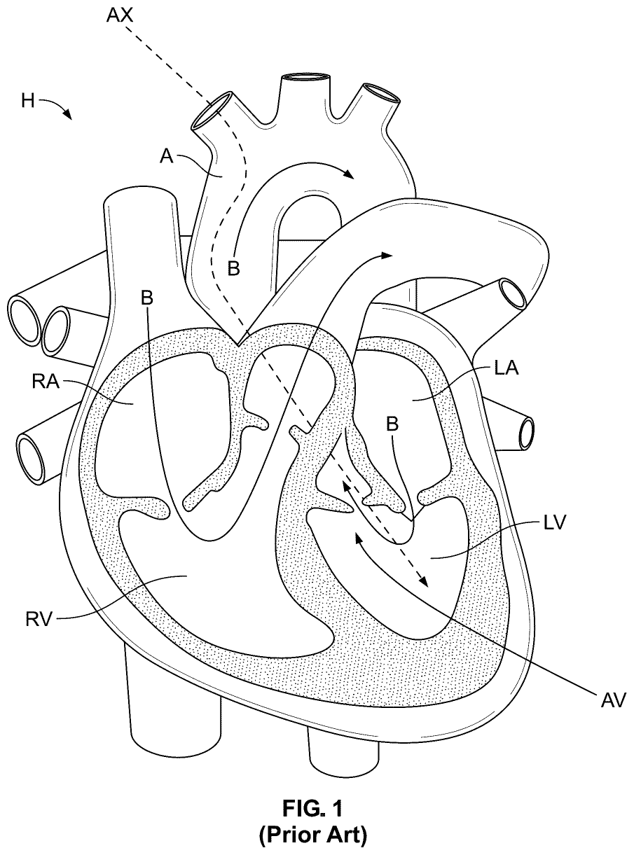

FIG. 1 is a schematic cutaway representation of a human heart H. The human heart includes two atria and two ventricles: right atrium RA and left atrium LA, and right ventricle RV and left ventricle LV. Heart H further includes aorta A. Disposed between left ventricle LV and aorta A is aortic valve AV. The aortic valve, also known as the left semilunar valve or the left arterial valve, generally includes three leaflets that coapt to regulate blood flow between left ventricle LV and aorta A. When left ventricle LV contracts during systole, aortic valve AV opens, and blood is pushed from the left ventricle through aorta A to major arteries of the vasculature system. Blood flows through heart H in the direction shown by arrows “B”.

A dashed arrow, labeled “AX”, indicates an approach of delivering an intracardiac pump to a target site via the axillary artery, in this case to a location across aortic valve AV. In such cases, an incision is made on the axillary artery and a graft is attached. The intracardiac blood pump is inserted into the graft, advanced through the axillary artery, into the target site through the aorta A. In some embodiments, an introducer sheath may be inserted into the graft. In such embodiments, the intracardiac blood pump may pass through the introducer sheath, through the axillary artery, and into the target site through the aorta A. In some embodiments, the intracardiac pump may be delivered via a more proximate vessel, such as the subclavian, aortic, or innominate. Echocardiography, fluoroscopy, and/or other imaging techniques may be used to help guide a delivery device, such as a catheter, to the target site. Other approaches are possible for delivering the intracardiac pump across aortic valve AV, such as a direct aortic approach, or to other target sites within heart H.

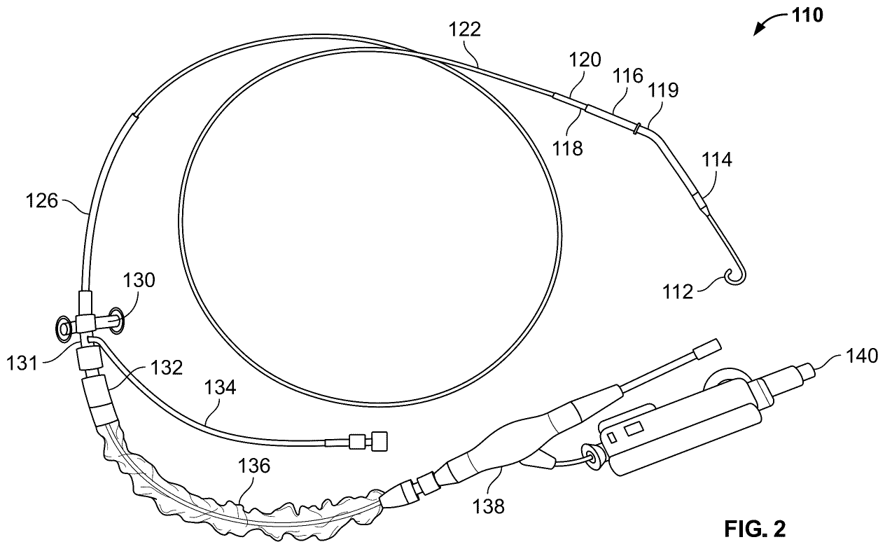

FIG. 2 illustrates an intracardiac pump and sheath assembly 110. A handle 138 may be provided at the proximal end of intracardiac pump and sheath assembly 110. Handle 138 may be operably coupled to the proximal end of a catheter 122 and, therefore, arranged to advance the catheter within the cardiovascular system of a patient and to retract the catheter from the cardiovascular system of the patient, unless movement of the catheter is prevented by a securement device 132, as will be explained in further detail hereinafter.

Catheter 122 may be enclosed by a protective sleeve 136 extending between handle 138 and securement device 132. Put differently, the proximal end of protective sleeve 136 may be attached to handle 138, and the distal end of the protective sleeve may be attached to securement device 132, to enclose and protect a proximal portion of catheter 122. Protective sleeve 136 may be formed of any material, such as a medical grade plastic, suitable for preventing the proximal end of catheter 122 from being contaminated as the catheter is advanced into the vasculature of a patient.

A hemostasis valve 131 may be provided between a distal end of securement device 132, and a proximal end of butterfly 130. Hemostasis valve 131, securement device 132, and butterfly 130 may be removably coupled from one another or manufactured as a single unitary component. A proximal end of a sheath 126 may be coupled to butterfly 130. Sheath 126 defines a lumen extending from the proximal end of the sheath to a distal end of the sheath that is sized to slidably receive catheter 122 therethrough.

Intracardiac pump and sheath assembly 110 may also include a purge fluid port 140. The purge fluid port 140 may be in communication with handle 138 and may be arranged to provide purge fluid (e.g., a solution of dextrose or glucose with heparin) to a purge lumen (not shown) within catheter 122. The purge lumen of catheter 122 may be in fluid communication with, and configured to deliver the purge fluid to, the motor housing 120 of the intracardiac pump. Motor housing 120 may include a motor and an impeller. In some embodiments, the motor may be external to the patient, in which case catheter 122 may enclose a flexible drive, such as a shaft or cable, and motor housing 120 may enclose an impeller connected to that drive shaft or cable.

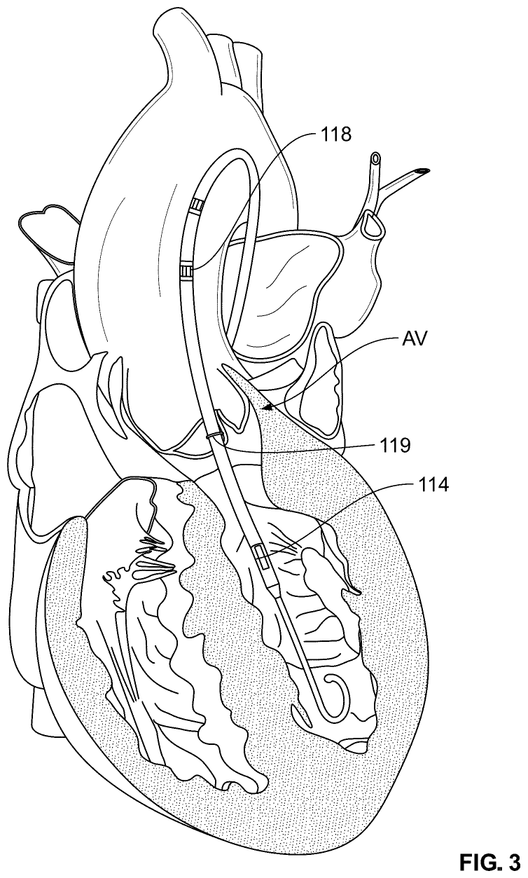

With continued reference to FIG. 2, the distal end of motor housing 120 may be coupled to a blood outflow cage 118. The distal end of blood outflow cage 118 may be coupled to a cannula 116, which in turn, may be coupled a proximal end of blood inflow cage 114. Cannula 116 may include a marking 119, such as a radiopaque marker that is visible under fluoroscopy, to assist a clinician in properly positioning the cannula within the native heart valve as shown in FIG. 3. In some embodiments, the cannula 116 may be expandable. A pigtail extension 112 may extend from the distal end of blood inflow cage 114. In this regard, when left ventricle LV contracts during systole, the left ventricular wall may contact pigtail extension 112, instead of blood inflow cage 114, thereby preventing intracardiac pump from damaging the ventricular wall. In other embodiments, the blood pump may not include a pigtail extension 112.

When the pump is operated, blood will be pumped in the proximal direction from blood inflow cage 114, through cannula 116, to blood outflow cage 118. In this respect, the intracardiac pump illustrated in FIG. 2 is designed for left heart support. The intracardiac pump, however, may alternatively be configured to pump blood in the distal direction (e.g., for applications where the pump is used for right heart support), in which case cage 118 would operate as the blood inflow cage, and cage 114 would operate as the blood outflow cage.

Intracardiac pump and sheath assembly 110 may be introduced into the vasculature of a patient via an introducer sheath assembly (not shown). In some embodiments, the intracardiac pump may be inserted into vasculature of the patient through a graft sutured to the cardiovascular system of a patient, in a chimney fashion. For example, when delivering an intracardiac pump using a percutaneous axillary artery approach, intracardiac pump and sheath assembly 110 may be inserted through a graft secured to the axillary artery.

Once sheath 126 has been fully inserted into the patient, the clinician may secure the sheath to the patient at or near the incision (e.g., adjacent the clavicle) using butterfly 130. In this regard, butterfly 130 may be affixed to the patient, using adhesives or sutures, to secure the sheath 126 and securement device 132 relative to the patient. With sheath 126 secured to the patient, the clinician may then advance the intracardiac pump to the target site and use securement device 132 to restrict further movement of the intracardiac pump after it has been properly positioned within heart H.

Securement device 132 may be a conventional Tuohy-Borst type device, although any other securement device known in the art may be used. Conventional Tuohy-Borst type devices include a barrel that may be rotated in a first direction (e.g., a clockwise direction) to clamp securement device 132 about catheter 122, which in turn restricts movement of the catheter. As mentioned herein, however, movement of the patient can adjust the positioning of catheter 122 between securement device 132 and the distal end of the catheter, which in turn, can cause migration of the intracardiac pump.

FIGS. 4-6 illustrate a graft 200 integrated with a catheter fixation device designed to secure catheter 122 within the graft and relative to the cardiovascular system of the patient. It will be appreciated that the intracardiac pump will be more stably secured within heart H as the distance between the catheter fixation device of graft 200 and the heart decreases. That is, reducing the length between the intracardiac pump and the location at which the catheter is fixed, will reduce the ability of the intracardiac pump to migrate. For this reason, securing catheter 122 to graft 200, and proximate heart H, will improve stability of the intracardiac pump compared to conventional external securement devices.

Graft 200 is primarily described herein in conjunction with intracardiac pump and sheath assembly 110. However, graft 200 may be used with any intracardiac blood pump, or other medical device, delivered into a patient by a delivery system, such as a catheter, when a clinician wishes to selectively restrict movement of that device. Examples of such systems include angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, or any other venous or arterial based introduced catheters and devices.

It will be appreciated that graft 200 is designed to perform a similar function to that of butterfly 130 and securement device 132 (e.g., securing sheath 126 to the patient and/or preventing movement of catheter 122 relative to the cardiovascular system of the patient). Consequently, in some embodiments graft 200 may be used with intracardiac pump and sheath assembly 110 as depicted in FIG. 2 (i.e., along with butterfly 130 and securement device 132), while in other embodiments, graft 200 may render the use of butterfly 130 and securement device unnecessary.

With reference to FIGS. 4-6, graft 200 may include a body 202 formed of a biocompatible material, such as a woven fabric, for example, Dacron or polytetrafluoroethylene. However, it will be appreciated that any other materials known in the vascular grafting art may be utilized. Body 202 may be generally tubular in shape and include a sidewall 204 extending from a proximal end 206 to a distal end 208 that defines a conduit 210 sized and configured to receive intracardiac pump and sheath assembly 110. The distal end 208 of body 202 may be designed to be sutured to the axillary artery in a chimney fashion.

Graft 200 may include one or more fixation devices 212 integrated into body 202 and configured to transition a portion of conduit 210 from a first diameter, in which catheter 122 is movable along the length of the conduit, to a second diameter smaller than the first diameter, in which the fixation device 212 clamps the catheter and secures the catheter within the conduit and relative to the cardiovascular system of the patient. As used herein, “integrated” or “integrally formed” means the one or more fixation device 212 are pre-operatively coupled to body 202, for example, during manufacturing of graft 200, or by a user, after the graft is manufactured and prior to the graft being sutured to the cardiovascular system of the patient. In this regard, fixation device 212 removes the need for a surgeon to separately apply other devices about the graft, or at other subcutaneous or external locations, to secure catheter 122 relative to the cardiovascular system and/or access site of the patient. Thus, graft 200 may expedite the surgical procedure and reduce complications associated with prolonged surgical procedure time.

With specific reference to FIG. 4, fixation device 212 may include a filament 214. In some embodiments, filament 214 may be formed of the same material as the body 202 of graft 200. In other embodiments, filament 214 may be formed of a different material, for example, a silk material. Filament 214 may be woven through the sidewall 204 of body 202 along a plane formed generally perpendicular to a longitudinal axis of conduit 210. In this regard, a first end 216 of filament 214 may be woven through sidewall 204 in a first direction, such as a clockwise direction, and a second end 218 of filament 214 may be woven through sidewall 204 in a second direction opposite to the first direction, such as a counter-clockwise direction. In this regard, filament 214 may be formed as a draw-string designed to cinch the sidewall 204 of body 202 when the first and second ends 216, 218 are tensioned. Put differently, tensioning the first and second ends of filament 214 will reduces the size of conduit 210 and compress the sidewall 204 of graft 200 about catheter 122, thereby preventing the catheter from moving along the length of the conduit.

In some embodiments, the first end 216 of filament 214 and the second end 218 of filament 214 may be tied, adhered, or otherwise secured in a knot, or a loop, to prevent the first and second ends of the filament from inadvertently slipping within sidewall 204. Nevertheless, in other embodiments, the first end 216 of filament 214 and the second end 218 of the filament need not be knotted or looped.

In use, graft 200 may be used in conjunction with an intracardiac pump and sheath assembly 110 to efficiently stabilize the intracardiac pump within heart H. Although use of graft 200 is described hereinafter in connection with a percutaneous intracardiac pump that is delivered to the left-side of the heart using an axillary approach, it will be appreciated that the graft 200 may be used in conjunction with percutaneous intracardiac devices providing right heart support, as well as other medical devices secured to a delivery system (e.g., a catheter) for which a clinician wishes to selectively restrict movement.

First, a physician may make an infraclavicular incision to provide an access point to the axillary artery of the patient. The distal end 208 of graft 200 may then be sutured to the axillary artery in a chimney fashion. With graft 200 secured to the axillary artery, the intracardiac pump may then be secured to the distal end of intracardiac pump and sheath assembly 110, advanced through graft 200, and into the axillary artery.

Under echocardiography and/or other traditional imaging techniques, the clinician may operate handle 138 to advance catheter 122 toward the target site by tracking the intracardiac pump into heart H via aorta A. As shown in FIG. 3, while using fluoroscopy, the physician may align the marking 119 on cannula 116 within the aortic valve AV for deployment in the left ventricle of a patient. Once deployed, blood inflow cage 114 may be centrally positioned within left ventricle LV and blood outflow cage 118 may be sufficiently positioned within aorta A.

After the clinician has confirmed that the intracardiac pump is properly positioned within the heart H of the patient, the clinician may tension the first end 216 of filament 214 and the second end 218 of filament 214 to reduce the size of the conduit 210, thereby crimping the sidewall 204 of the graft about an exterior surface of catheter 122 and securely fixing the catheter within the graft and relative to the cardiovascular system of the patient. In some embodiments, when filament 214 is formed as a draw-string, the filament will not release tension after the first and second ends 216, 218 have been tensioned. The surgeon may nevertheless optionally tie the first and second ends of filament 214 in a knot after tensioning the draw-string. In embodiments, in which the filament is not formed as a draw-string, the surgeon may tie the first and second ends of filament 214 in a knot, or otherwise secure the first and second ends using a clamp or other means to stabilize catheter 122 within graft 200.

It will be appreciated that the physician may optionally tension other filaments 214 located along the length of graft 200 for additional stability if desired. For example, in some embodiments, a first filament 214 may be provided near the distal end 208 of body 202 to secure catheter 122 at a location close to the axillary artery, and a second filament 214 may be provided adjacent the proximal end 206 of the body to provide stability at a location adjacent to the access site. In other embodiments, graft 200 may be provided with a single filament located at any location along a length of the graft, or two or more filaments spaced apart from one another at any location along the length of the graft.

With the intracardiac pump stabilized across the aortic valve AV, the intracardiac pump may be turned on to suction blood from the left ventricle LV into blood inflow cage 114, through connecting cannula 116, and out from blood outflow cage 118 into aorta A. Thereafter, the incision may then be closed.

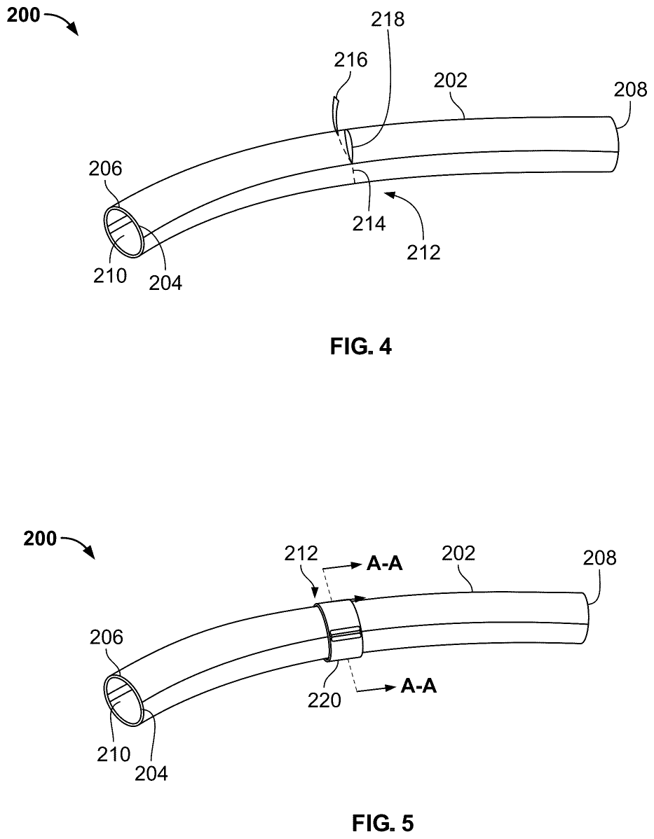

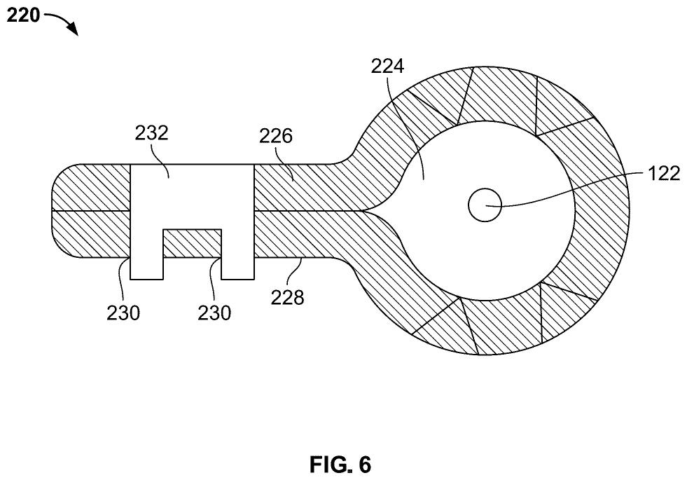

Turning now to FIGS. 5 and 6, graft 200 is illustrated with a fixation device 212 according to another embodiment of the present disclosure. As depicted in FIGS. 5 and 6, fixation device 212 may be a clamp 220 integrated into graft 200. Clamp 220 may include a ring 224 that forms a portion of conduit 210 and first and second wings 226, 228 provided about ring 224. In some embodiments, ring 224 may extend continuously about a circumference of conduit 210. For example, ring 224 may be coupled about an external surface of the sidewall 204, an internal surface of the sidewall, or form a portion of the sidewall of graft 200. In other embodiments, ring 224 may discontinuously extend about a circumference of conduit 210 such that the combination of the ring and sidewall 204 form a portion of the conduit. In some embodiments, the thickness of ring 224 may be equal to the thickness of the sidewall 204 of body 202. In other embodiments, the thickness of the ring 224 may be different than the thickness of the sidewall 204 of body 202. For example, the thickness of ring 224 may be greater than the thickness of sidewall of body 202.

First and second wings 226, 228 may be integrally formed about ring 224. In other examples, first and second wings 226, 228 may be positioned about ring 224 intraoperatively. In either case, ring 224 is designed to reduce in size when first and second wings 226, 228 are clamped together to secure catheter 122 within the ring 224 and, in turn, relative to graft 200.

Ring 224 may be formed of a first material and first and second wings 226, 228 may be formed of a different material for performing different function. For example, ring 224 may be formed of a substantially resistant material, such as a polymer, and more particularly a rubber or a silicone. In this regard, ring 224 may designed to compresses catheter 122 while distributing the compressible load to prevent damage. However, it will be appreciated that ring 224 may be formed of any suitable material. In some examples, an innermost surface of ring 224 may be textured to increase friction and improve catheter fixation.

Wings 226, 228 may be formed of a substantially rigid material, such as stainless steel, or another medical grade metal or metal alloy. In this regard, the substantially rigid material of wings 226, 228 may provide and maintain a clamping force on ring 224 to maintain the ring in a clamped position about catheter 122. Nevertheless, wings 226, 228 need not be limited to a substantially rigid material, and wings 226, 228 may be formed of any suitable material.

As depicted in FIG. 6, wings 226, 228 may extend from ring 224 in the same direction and define one or more apertures 230 for receiving a fastening device such as a surgical staple 232, a suture, or the like. When stapled, the staple 232 is designed to pass through the aperture(s) 230 to clamp first and second wings 226, 228, which in turn, compresses ring 224 and secures catheter 122 within the body 202 of graft 200. In other embodiments, wings 226, 228 need not include apertures 230 and may instead be clamped together and secured via an adhesive or any other suitable means.

In use, a surgeon may operate graft 200 provided with clamp 220 to securely stabilize intracardiac pump and sheath assembly 110 within heart H in the same manner as previously described with respect to the embodiment referenced in FIG. 4 but for the manner in which clamp 220 is actuated. For this reason, only this aspect of the procedure will be described hereinafter.

After the clinician has confirmed that the intracardiac pump is properly positioned within the heart H of the patient, a surgical staple 232 may be inserted through aperture(s) 230 to clamp first and second wings 226, 228. As first and second wings 226, 228 are clamped together, the rigid material may compress ring 224, thereby reducing the circumferential size of ring 224 around catheter 122. In this regard, when first and second wings 226, 228 are clamped, clamp 220 may simultaneously secure catheter 122 within graft 200. Consequently, catheter 122 may be stabilized within the conduit 210 of graft 200 and relative to the cardiovascular system and/or the access of the patient. Alternatively, a clinician may use a suture, adhesive, or another mechanism to clamp first and second wings 226, 228. However, it will be appreciated that stapling may expedite the surgical procedure and remove user variability, compared to suturing.

It will be appreciated that the structure of clamp 220 is merely exemplary and any clamp, clasp, zip-tie, compressible ring, or similar device may be integrated into graft 200 for clamping the sidewall 204 of graft 200 about catheter 122, thereby securing the catheter relative to the cardiovascular system of the patient and/or relative to the access site.

As disclosed herein, graft 200 offers several advantageous over traditional external securement devices. A previously mentioned, when catheter 122 is externally secured, movement of the patient may result in movement of the catheter, which in turn, may result in migration of the intracardiac pump. Graft 200 permits catheter 122 to be stabilized subcutaneously (e.g., closer to the heart) and reduces catheter 122 motion due to patient movement. Furthermore, securing the catheter 122 within graft 200 removes the need for a surgeon to perform subsequent securement steps and reduces surgical time.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A catheter fixation device, comprising:
a graft having a proximal end, a distal end, and a sidewall defining a conduit having a length extending between the proximal end and the distal end; and
a fixation device integrated into the graft, the fixation device being configured to transition a portion of the conduit from a first diameter, in which a catheter is movable along the length of the conduit, to a second diameter smaller than the first diameter, in which the catheter is fixed within the conduit.

2. The device of claim 1, wherein the fixation device comprises a filament.

3. The device of claim 2, wherein the filament is weaved within the sidewall.

4. The device of claim 2, wherein the filament is arranged as a draw-string arranged to cinch the portion of the conduit.

5. The device of claim 1, wherein the fixation device comprises a clamp.

6. The device of claim 5, wherein the clamp comprises a ring formed of a resilient material and wings formed of a rigid material, the wings being arranged to clamp the ring.

7. An assembly, comprising:
a catheter for delivering a medical device into a cardiovascular system of a patient; and
a graft having a proximal end, a distal end, a sidewall defining a conduit having a length extending between the proximal end and the distal end, and a fixation device integrated into the graft, the fixation device being configured to transition a portion of the conduit from a first diameter, in which the catheter is movable along the length of the conduit, to a second diameter smaller than the first diameter, in which the catheter is fixed within the conduit.

8. The assembly of claim 7, further comprising the medical device, the medical device being an intracardiac pump.

9. The assembly of claim 7, wherein the fixation device comprises a filament.

10. The assembly of claim 9, wherein the filament is weaved within the sidewall.

11. The assembly of claim 9, wherein the filament is arranged as a draw-string arranged to cinch the portion of the conduit.

12. The assembly of claim 7, wherein the fixation device comprises a clamp including a ring formed of a resilient material and an outer rigid material arranged to clamp the ring.

13. The assembly of claim 12, wherein the outer rigid material includes first and second wings.

14. The assembly of claim 12, wherein the outer rigid material defines one or more apertures configured to receive a fastening device.

15. A method of stabilizing a medical device within a cardiovascular system of a patient, the method comprising step of:
tracking a catheter, carrying the medical device, through a conduit of a graft integrated with a fixation device and into the cardiovascular system of the patient;
positioning the medical device at a target site within the cardiovascular system of the patient; and
actuating the fixation device to transition a portion of the conduit from a first diameter, in which the catheter is movable along a length of the conduit, to a second diameter smaller than the first diameter, in which the catheter is fixed within the conduit and relative to the cardiovascular system of the patient.

16. The method of claim 15, wherein the medical device is an intracardiac pump and the target site is across a native heart valve.

17. The method of claim 15, further comprising:
creating an incision through skin of the patient adjacent a clavicle of the patient.

18. The method of claim 15, wherein the fixation device comprises a filament, and the actuating step comprises tensioning the filament.

19. The method of claim 15, wherein the fixation device comprises a resilient ring and an outer rigid material, and the actuating step comprises applying a clamping force to the outer rigid material.

20. The method of claim 19, wherein the applying the clamping force comprises applying a staple through an aperture of the outer rigid material to compress the resilient ring.
